SEQUENCE LISTING (5 sequences)

SEQ ID NO: 1 - DNA, 20 bp, Artificial sequence
  Forward primer
agagtttgatcctggctcag

SEQ ID NO: 2 - DNA, 21 bp, Artificial sequence
  Reverse primer
acggttaccttgttacgactt

SEQ ID NO: 3 - DNA, 18 bp, Artificial sequence
  Forward primer
caccgagattcaacatgg

SEQ ID NO: 4 - DNA, 24 bp, Artificial sequence
  Reverse primer
cccactgctgcctcccgtaggagt

SEQ ID NO: 5 - DNA, 1007 bp, Bacteria
  Lactobacillus paracasei subsp. paracasei SG96 strain
ggggcgcgtgctatacatgcaagtcgaacgagttctcgttgatgatcggtgcttgcaccgagattcaacatggaacgagtggcggacgggtgagtaacacgtgggtaacctgcccttaagtgggggataacatttggaaacagatgctaataccgcatagatccaagaaccgcatggttcttggctgaaagatggcgtaagctatcgcttttggatggacccgcggcgtattagctagttggtgaggtaatggctcaccaaggcgatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagcgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtggttgggagggtttccgcccttcagtgccgcagctaacgcattaagcattccgcctgggggagtacgaccgcaaggttgaactcaaggaattgacgggggcccgcacaagcggtggagcatgtggtttattcgaagcaacgcgagagcttaccagtcttgacatcttttgatcactgagagatcagttt

(19) United States
(12) Patent Application Publication
(10) Pub. No.: US 2010/0196341 A1
(43) Pub. Date: Aug. 5, 2010

(54) Novel Lactobacillus paracasei subsp. paracasei SG96, a Bacteriostatic Composition Containing the same and Use Thereof

(75) Inventors: Yu-Shan WEI, Tainan County (TW); Fang-Yun TAI, Kaohsiung City (TW)

Correspondence Address: SCHMEISER OLSEN & WATTS, 18 E UNIVERSITY DRIVE, SUITE # 101, MESA, AZ, 85201, US

(73) Assignee: SYNGEN BIOTECH CO., LTD., Tainan County (TW)

(21) Appl. No.: 12/366,515

(22) Filed: Feb. 5, 2009

(51) Int. Cl. A61K 35/74; C12N 1/20

(52) U.S. Cl. 424/93.45; 435/252.9

(57) ABSTRACT

A novel Lactobacillus paracasei subsp. paracasei SG96, a bacteriostatic composition containing the same and use thereof, comprise a novel Lactobacillus paracasei subsp. paracasei SG96 strain, wherein said strain had been deposited in China General Microbiological Culture Collection Center, CGMCC, accession number: CGMCC 2697, characterized in that said strain can inhibit the growth of pathogenic bacteria such as Escherichia coli, Salmonella typhimurium and the like, and even has an effect of killing bacteria; and a bacteriostatic composition containing said Lactobacillus paracasei subsp. paracasei SG96 strain, characterized in that said composition can be used as an additive in animal drinking water, as an additive in animal feed, as an medical composition for animals and humans, as an additive in food products as an additive in beverages, and as a food product, beverage, and health food products, and the like.

### BACKGROUND OF THE INVENTION

1. Field of the Invention

The invention relates to a novel Lactobacillus paracasei subsp. paracasei SG96 strain, and in particular, to a novel Lactobacillus paracasei subsp. paracasei microbial strain that can inhibits pathogenic bacteria such as Escherichia coli, Salmonella typhimurium, and the like, a bacteriostatic composition containing said Lactobacillus paracasei subsp. Paracasei and use of said composition.

2. Description of the Prior Art

In the past several decades, since the population has quickly increased and the level of material life raised significantly, people's demand for meat products has greatly increased. In order to satisfy market demand, the raising of numerous livestock animals has gradually been scaled up. Especially, in developing countries, because of their limited ability to import food, economic animals were raised en masse under conditions of high animal density. This situation resulted in rapid dissemination of a number of diseases among animals. To solve the problem of sickness among livestock or to promote the fast growth of livestock, historical approaches adopted were addition of antibiotics into animal feeds, or administration of antibiotics into animals, which has lead to generations of tremendously drug-resistant strains of disease.

In late 1960 decade, the introduction of antibiotics caused the human race to obtain the unprecedented victory in the field of treating bacterial infection. A number of originally fatal infectious diseases became no longer a threat. However, the emergence of drug-resistant bacteria in recent years had elevated once more the anxiety and fear of mankind to infectious diseases, since antibiotics are no longer a catholicon for controlling diseases. Further, the speed in which drug-resistant bacteria develop and spread have surpassed our imagination.

Bacterial drug-resistance has brought about not only lowered treatment efficacy and even ineffectiveness, but also a serious influence on human health. Forty years ago, about 7 million humans died due to infectious diseases worldwide every year, while at present, in spite of the great advances in medical science, the annual death due to infectious diseases has dramatically increased to 20 million per year. As people take in long term residual antibiotics-contained in food products such as meats, eggs, milks and the like derived from poultry and livestock, these residual antibiotics result in adverse effect on human body, and may accumulate in the body so as to cause continual occurrences of drug-resistant strains.

In the past decade, meat product safety issues have raised concerns among consumers, fishing and livestock producers, as well as government. The constant high level of concern over meat products by consumers have come from several terrifying food safety events in domestic and foreign countries in last several years, such as mad cow disease (Bovine Spongiform Encephalopathy) (BSE) in England, H5N1 avian flu in southeast Asia and mainland China and the like. In consideration meat products safety, worldwide attention has focused on the adverse consequences caused by residual antibiotics. In order to delay the occurrence of drug-resistance strains, in the mid-1990's, some European countries started to restrict and control the use of antibiotics in medicine and agriculture, in an effort to lower the evolution of drug-resistance strains. Since 1995, the European Union had forbidden the use of some antibiotics in feeds. These approaches achieved good results, for example, researchers in Danish Veterinary Laboratory, Copenhagen indicated that after forbidding the use of avoparcin antibiotic, the drug-resistance to avoparcin by Enterococcus faecium in digestive tract of Danish chicken reduced from 73% in 1995 down to 6% in 2000. After banning the use of avoparcin for 5 years of the same time period, Germany and Holland demonstrated that drug-resistance strains in animals and humans had reduced. Consequently, the European Union banned the addition of antibiotics in feeds since January 2006.

The purpose of adding antibiotics in feeds is to promote growth, reduce coarse feed, and enhance growth efficiency of poultry and livestock. However, since the vegetative cycle of the poultry and livestock is shortened, the absorption of nutritive substances is inevitably reduced. Consequently, the unique flavor and quality of the poultry and livestock products will be lower, and more seriously, residual drugs bring about serious hazards to human health, and especially, the health of growing children. Therefore, forbidding the use of antibiotics or gradually reducing the types and dosages of antibiotics in livestock feed is becoming a worldwide trend. However, the stepwise banning of antibiotics may derive many problems, and in regards to normal production in animal husbandry, producers have accepted the concept of using probiotic agents as the substitute for antibiotics and feed additives.

Probiotics are predominantly lactic acid bacteria. Lactic acid bacteria can maintain and restore the balance of bacterial colonies in the digestive tract of poultry and livestock, reduce stress, enhance immunological and disease-resistant abilities, and decrease morbidity. Furthermore, silage obtained from lactic acid bacteria fermentation, and liquid fermented feed are advantageous, since lactic acid bacteria fermented feed not only can effectively retain the nutritive ingredients in the feed and raise digestion rates, but also can, during digestion in intestinal tract, eliminate pathogenic bacteria as well as kill certain microorganisms that may induce diseases both in humans and animals. Lactic acid bacteria had been acknowledged generally as a very ideal substitute for antibiotics.

Beneficial effects of probiotics in the bodies of poultry and livestock include:
1. Maintaining and restoring the balance of bacterial colonies taking lactic acid bacteria as a superior bacterial colony in the digestive tract of poultry and livestock; reducing the stress state of poultry and livestock (stress, such as weaning, changing feed, high temperature, chill, transportation, frightening, disease and the like), and further eliminating the environmental disorder in the digestive tract; suppressing the abnormal proliferation of harmful bacteria as well as the infiltration and anchoring of pathogenic bacteria; Preventing and treating diseases in the digestive tract such as scours, diarrhea, and the like; reducing or eliminating the generation of endotoxin; and especially remarkable, the effect of supplementing lactic acid bacteria for young animal and poultry that have not established their microorganism system.
2. Producing non-specific immunological regulatory factor; enhancing the immunological disease-resistant ability; decreasing disease infection rate and death rate; effectively replacing antibiotics and chemical drugs; reducing residual effects of drugs; and promoting food quality for human consumption.
3. Supplementing nutritive ingredients; promoting growth of poultry and livestock; inducing the generation of digestive enzymes; enhancing abilities of digestion and absorption; and markedly increasing the output and benefit of animal husbandry.
4. Reducing odorous substances in the excrements of poultry and livestock; and improving the environment of the farm.
5. Increasing the output of milk, meat and eggs; adding to the benefit to animal husbandry.

Safety problems with food products have received high attention from various governments worldwide. At present, governments are positively promoting green animal husbandry and assisting with the development of green products in terms of regulatory policies, the financial resources, and the material resources. There is a need for green farming, to develop a brand of green livestock products, to give operators of green products a material benefits, and to supply consumers with high quality, safe poultry and livestock food products so as to benefit humanity.

### SUMMARY OF THE INVENTION

One object of the invention is to provide a novel Lactobacillus paracasei subsp. paracasei SG96 strain. Said strain is significantly different in phylogenetic relation to conventional Lactobacillus paracasei BCRC 910220, Lactobacillus paracasei subsp. paracasei BCRC14001, and Lactobacillus paracasei subsp. paracasei BCRC16100, and hence is a novel Lactobacillus paracasei subsp. paracasei microbial strain.

Another object of the invention is to provide a bacteriostatic composition containing said novel Lactobacillus paracasei subsp. paracasei SG96, characterized in that said composition has an effect of inhibiting the growth of pathogenic bacteria such as Escherichia coli, Salmonella typhimurium and the like.

Yet another object of the invention is to provide the use of said bacteriostatic composition containing said novel Lactobacillus paracasei subsp. paracasei SG96, characterized in that said bacteriostatic composition can be used as an animal feed, and as additives in drinking water, to enhance the pathogenic bacteria-resistant ability of animals; and that said bacteriostatic composition can be further used as human food products, and as additives in beverages or food products, so as to enhance the pathogenic bacteria-resistant ability in the human body.

The novel Lactobacillus paracasei subsp. paracasei SG96, a bacteriostatic composition containing the same and use thereof that can achieve those above-described objectives of the invention comprise: a novel Lactobacillus paracasei subsp. paracasei SG96 strain, said strain is obtained by screening from pig intestinal tract excreta, and after identification through Gram stain analysis, API 50 CHL analysis, 16S rDNA sequencing analysis, and phylogenetic analysis, it is confirmed to be a novel Lactobacillus paracasei subsp. paracasei microbial strain. Said Lactobacillus paracasei subsp. paracasei SG96 strain has been deposited in China General Microbiological Culture Collection Center (CGMCC), with accession number as CGMCC 2697, on Oct. 9, 2008.

As Lactobacillus paracasei subsp. paracasei SG96 strain according to the invention is subjected to disc-agar diffusion analysis and mixed culture experiment analysis with pathogenic bacteria, respectively, results indicated that the inventive SG96 strain can suppress the growth of pathogenic bacteria such as E. coli, S. typhimurium and the like, and even has an effect of killing bacteria.

When the inventive Lactobacillus paracasei subsp. paracasei SG96 strain is formulated into aqueous solution and fed to the un-weaned piglet, the result indicated that the inventive SG96 strain can promote the weight gain of the un-weaned piglet, improve the feed conversion rate of the piglet feed, have good anti-diarrhea effect, exhibit performance better than that of conventional antibiotics, and demonstrated that the inventive SG96 strain can enhance pathogenic bacteria-resistant ability of the animal.

In addition to being formulated into aqueous solution to be used as drinking water for animals, the anti-bacteria composition containing the inventive Lactobacillus paracasei subsp. paracasei SG96 strain can be used in conjunction with conventional feed additive preparation techniques and formulated into other forms, including animal feed additives, medical composition for animals and humans, additive for food products, additive for beverages, as a food product, beverage, health food product and the like, for oral administration by animal and humans. In a preferred embodiment, said anti-bacteria composition can be processed into lactic acid tablets, powder or granules, and can be added into animal feed or food products (for example: 1 Kg of powder or granular Lactobacillus paracasei subsp. paracasei SG96 strain in 1 ton of feed (10⁷ CFU/g)); or can be used alone or in combination with other lactic acid bacteria to ferment milks into milk products such as yogurt milk or yogurt.

These features and advantages of the present invention will be fully understood and appreciated from the following detailed description of the accompanying Drawings.

### BRIEF DESCRIPTION OF THE DRAWINGS

FIG. 1 is the morphology graph of the inventive Lactobacillus paracasei subsp. paracasei SG96 strain;

FIG. 2 is an agarose gel electrophoresis pattern of a PCR-amplified 16S rDNA fragment of said Lactobacillus paracasei subsp. paracasei SG96 strain;

FIG. 3 is a sequence alignment chart of 16S rDNA among Lactobacillus paracasei subsp. paracasei strain SG96, BCRC 910220, BCRC14001, and BCRC16100;

FIG. 4 is a phylogenetic analysis chart of Lactobacillus paracasei subsp. paracasei strain SG96, BCRC 910220, BCRC14001, BCRC16100 and other Lactobacillus paracasei subsp. paracasei strain;

FIG. 5 shows the inhibition effect of Lactobacillus paracasei subsp. paracasei SG96 strain on Escherichia coli by using disc-agar diffusion analysis;

FIG. 6 shows the inhibition effect of Lactobacillus paracasei subsp. paracasei SG96 strain on Salmonella typhimurium by using disc-agar diffusion analysis;

FIG. 7 shows the result of mixed culture experiment of Lactobacillus paracasei subsp. paracasei SG96 strain (10⁸ bacteria count) with pathogenic bacteria;

FIG. 8 shows the result of mixed culture experiment of Lactobacillus paracasei subsp. paracasei SG96 strain (10⁷ bacteria count) with pathogenic bacteria;

FIG. 9 shows the result of mixed culture experiment of Lactobacillus paracasei subsp. paracasei SG96 strain (10⁶ bacteria count) with pathogenic bacteria;

FIG. 10 shows the result of mixed culture experiment of Lactobacillus paracasei subsp. paracasei SG96 strain (10⁵ bacteria count) with pathogenic bacteria;

FIG. 11 shows the result of mixed culture experiment of Lactobacillus paracasei subsp. paracasei SG96 strain (10⁶⁴ bacteria count) with pathogenic bacteria;

FIG. 12 shows the effect of Lactobacillus paracasei subsp. paracasei SG96 strain on the weight change of un-weaned piglet; and

FIG. 13 shows the effect of Lactobacillus paracasei subsp. paracasei SG96 strain on the diarrhea of un-weaned piglet.

### DETAILED DESCRIPTION OF THE PREFERRED EMBODIMENT

The invention will be illustrated by way of the following examples, but the invention will not be limited thereto.

### EXAMPLE 1

### The Screening and Isolation of Lactobacillus paracasei subsp. paracasei SG96 Strain

Intestinal tract excreta were collected from pigs, placed in MRS broth medium (Difco™, REF288130), and cultured at 37° C. under anaerobic condition for 24 hours. Then, the resulting culture suspension was applied over a MRS agar plate (Difco™, REF288210), and incubated at 37° C. for 3 days. Thereafter, colonies on the agar medium were collected to obtain 1000 acid forming bacteria strains, which were screened by disc-agar diffusion to obtain lactic acid bacteria having inhibiting activities against E. coli (BCRC11634) and S. typhimurium (BCRC129407). A functional Lactobacillus paracasei subsp. paracasei SG96 having best activity was selected from these lactic acid bacteria. This strain had following culture characteristics: forming a grey colony of 2-3 mm diameter on MRS agar plate, and the best culturing temperature: 37° C. Said Lactobacillus paracasei subsp. paracasei SG96 had been deposited in China General Microbiological Culture Collection Center (CGMCC), with an accession number: CGMCC 2697, at Oct. 9, 2008.

### EXAMPLE 2

### Gram Stain Analysis of Lactobacillus paracasei subsp. paracasei SG96 Strain

The characteristics of the strain obtained in Example 1 were tested by Gram stain assay. The inventive strain belonged to Gram-positive bacteria, non-sporing anaerobes, and non-mobility bacteria, which were typical characteristics of Lactobacillus.

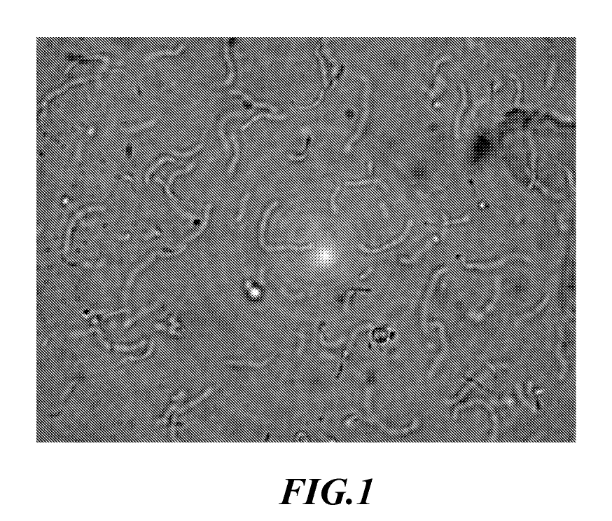

Said Lactobacillus paracasei subsp. paracasei SG96 strain has following bacteriological characteristics:
(a) Morphological characteristics:
   (1) Cell shape and size: When the cell was cultured in MRS broth medium at 37° C. under anaerobic condition for 24 hours, a bacillus present as rod-shape could be observed under microscopic examination (see FIG. 1).
   (2) Mobility: non-mobility
   (3) Flagellum: none
   (4) Sporogenesis: non-sporing
   (5) Gram stain: positive
(b) Culturing characteristics:
   (1) Medium: MRS broth medium, pH=6.25
   (2) Culturing condition: 37° C. under anaerobic condition
(c) Physiological characteristics:
   (1) Catalase: negative
   (2) Oxidase: negative
   (3) API 50 CHL test: see Example 3.

### EXAMPLE 3

### 16S rDNA Sequencing Analysis of Lactobacillus paracasei subsp. paracasei SG96 Strain

### 1. DNA Extraction

DNA was purified using FavorPrep™ Blood Genomic DNA Extraction Mimi Kit. The bacteria were cultured overnight. 200 μL of bacteria liquor was mixed with 20 μL proteinase K (proteinase K, before use, 110 μL ddH₂O was added and mixed by shaking for 5 min, to a concentration of 11 mg/mL, and stored at −20° C. till use), together with 200 μL FABG buffer, by shaking for 5 seconds. Then, the mixture was heated at 60° C. for 15 minutes and was centrifuged briefly. 200 μL 95% ethanol was added and mixed by shaking for 10 seconds. After brief centrifuge, the mixture was charged into a FABG column. The FABG column was placed in a Collection Tube, and centrifuged at 10,000 rpm for 2 minutes. The FABG column was then removed, the subnatant was decanted and after sucked dried with a tissue paper, the FABG column was returned into the Collection Tube. 500 μL W1 buffer (8 mL of 95% ethanol was added during the first use) was added, and centrifuged at 10,000 rpm for 2 minutes. Then, the FABG column was removed, and the subnatant was decanted and sucked dried with a tissue paper. The FABG column was returned again in the Collection Tube. 750 μL WB (40 mL of 95% ethanol was added during the first use) was added, and centrifuged at 10,000 rpm for 2 minutes, and the subnatant was decanted and sucked dried with a tissue paper. The FABG column was returned in the Collection Tube, and centrifuged at 10,000 rpm for 6 minutes. Thereafter, the resulting FABG column was placed in a 1.5 ml centrifuge tube. 150 μL of Elution Buffer was added, stood still for 3 minutes, and centrifuged at 10,000 rpm for 4 minutes. A pure DNA was obtained and stored at −20° C. till use.

2. The Amplification of 16S rDNA PCR Fragment

DNA of Lactobacillus paracasei subsp. paracasei SG96 strain, and DNA of Lactobacillus paracasei GMNL-32 strain (this strain had an accession number of BCRC 910220, was described in ROC Patent No. 1284149, and was a Lactobacillus paracaseis strain having a function for treating allergy-related condition), were subjected separately to amplification of 16S rDNA (ribosomal DNA) fragment. Primes used were prokaryotic 16S rDNA PCR universal primers that had been designed based on sequences of 8-27 bases and 1510-1492 bases of E. coli 16S rDNA gene (William G. Weisburg, Susan M. Barns, Dale A. Pelletier, and David J. Lane. 16S Ribosomal DNA Amplification for Phylogenetic Study. J. Bacteriol. January 1991; 173(2): 697-703), and had the following sequences:

| | |
|---|---|
| Forward primer fD1: | |
| 5′-AGAGTTTGATCCTGGCTCAG-3′ | (SEQ ID No: 1) |
| Reverse primer rP1: | |
| 5′-ACGGTTACCTTGTTACGACTT-3′ | (SEQ ID No: 2) |

Said 16S rDNA PCR fragment amplification was carried out with the following process: (1) 95° C. 5 minutes; (2) 94° C. 1 minute; (3) 60° C. 30 seconds; (4) 72° C. 1.5 minutes; (5) 72° C. 10 minutes, wherein steps (2) to (4) were repeated for 30 cycles.

Further, DNA of Lactobacillus paracasei subsp. paracasei BCRC14001 and DNA of Lactobacillus paracasei subsp. paracasei BCRC16100 were subjected separately to amplification of 16S rDNA fragment. Primers used were derived from 16S rRNA V1 region of Lactobacillus paracasei, and 16S rRNA conserved sequence of Lactobacillus (Ward, L. J. H., and Timmins, M. J. 1999. Differentiation of Lactobacillus casei, Lactobacillus paracasei and Lactobacillus rhamnosus by polymerase chain reaction. Lett. Appl. Microbiol. 29: 90-92.), and had following the sequences:

| | |
|---|---|
| Forward primer: | |
| 5′-CACCGAGATTCAACATGG-3′ | (SEQ ID No: 3) |
| Reverse primer: | |
| 5′-CCCACTGCTGCCTCCCGTAGGAGT-3′ | (SEQ ID No: 4) |

Said 16S rDNA PCR fragment amplification was carried out with the following process: (1) 95° C. 5 minutes; (2) 94° C. 1 minute; (3) 60° C. 30 seconds; (4) 72° C. 1.5 minutes; (5) 72° C. 10 minutes, wherein steps (2) to (4) were repeated for 30 cycles.

3. Sequence Alignment of 16S rDNA PCR Fragments

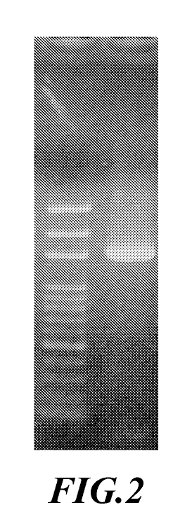

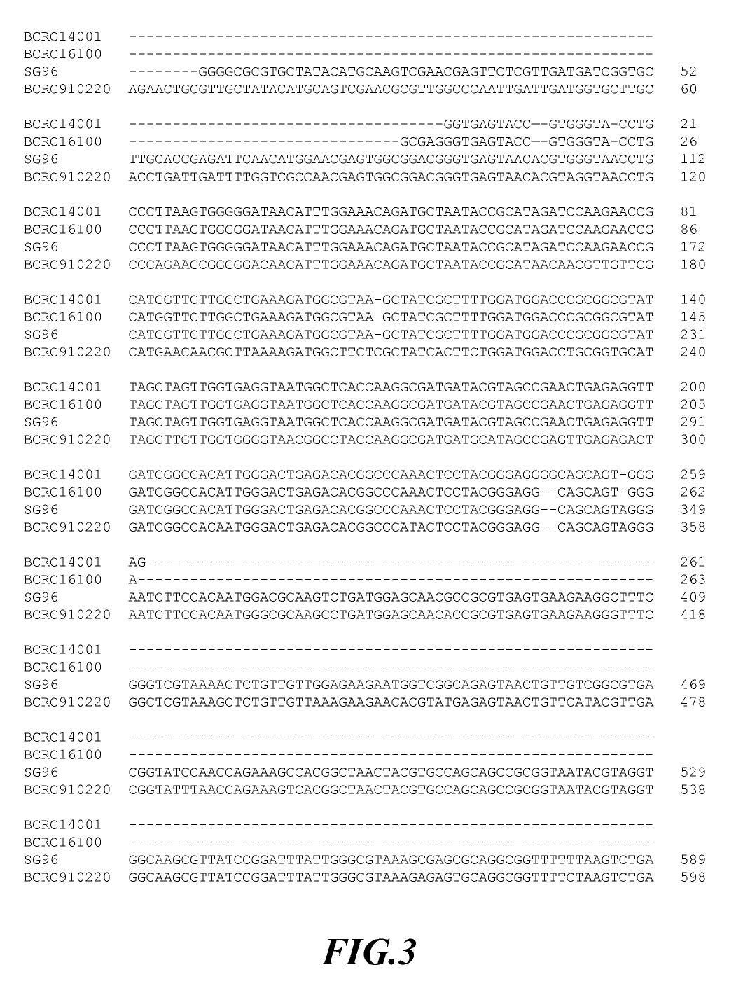

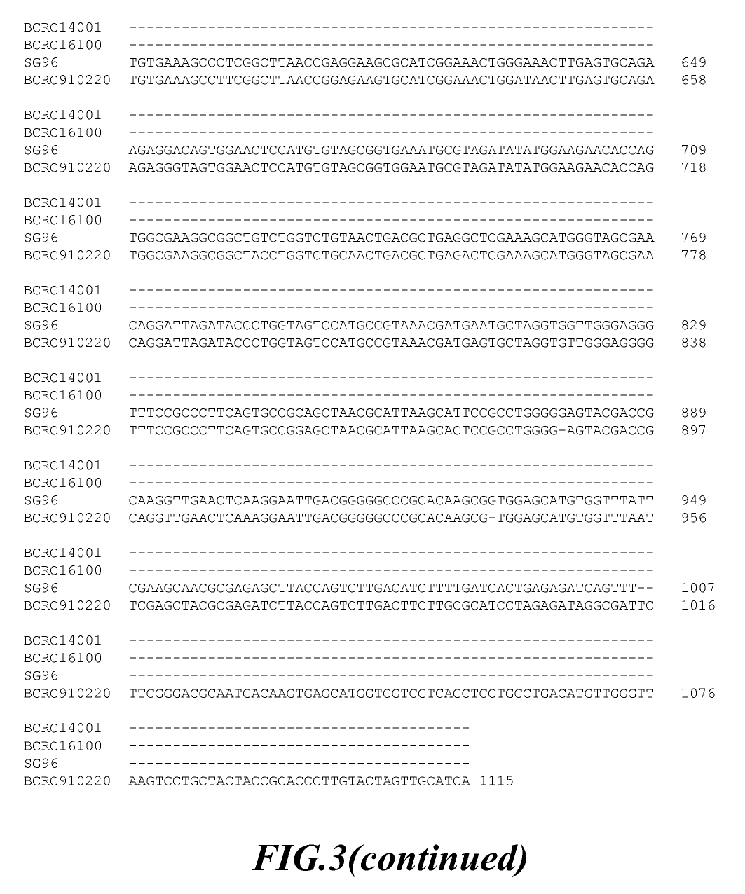

PCR product of 16S rDNA from Lactobacillus paracasei subsp. paracasei SG96 strain was subjected to agarose gel electrophoresis, with a result shown in FIG. 2. It was known from FIG. 2 that said fragment of PCR product had a size of about 1,500 bp (lengths of fragment standards were in order from top to bottom as follow: 3k, 2k, 1.5k, 1K, 900, 800, 700, 600, 500, 400, 300, 200, 100 bp). In addition, sequencing of the fragment was carried out and obtained the sequence of the 16S rDNA fragment from Lactobacillus paracasei subsp. paracasei SG96 strain as shown in SEQ ID No: 5. Sequence alignment of said sequence with multiplex sequence alignment data bank (NCBI blastn, http://www.ncbi.nlm.nih.gov/BLAST) was performed, with the results as shown in FIG. 3, and indicated that the sequence alignment result was in 98% precision as Lactobacillus paracasei.

### EXAMPLE 4

### API 50 CHL Analysis of Lactobacillus paracasei subsp. paracasei SG96 Strain

API 50 CHL test was used to examine the carbohydrate metabolism performance of the inventive lactic acid bacteria strain. API 50 CHL reagent could be used to identify the difference on Genius or Species among strains. The examination was performed by Food Industry Research and Development Institute, Hsinchu, Taiwan, ROC, and the result of API 50 CHL analysis thus obtained is shown in Appendix 1 and reproduced in Table 1. The result indicated that there was a homology of 98% in carbohydrate metabolism activities between the inventive strain and Lactobacillus paracasei subsp. paracasei 1, and accordingly, the API 50 CHL analysis confirmed further that the inventive strain is classified as Lactobacillus paracasei subsp. paracasei.

TABLE 1
Results of API 50 CHL analysis

Reference: 97ID036-L27
Strip: API 50 CHL V5.1
Profile: −−−−−+−−−−+ +++−−−−++− +++++++?−− ++−+−−−−−+ −+−−−−−−−

| | | | | | | | | | |
|---|---|---|---|---|---|---|---|---|---|
| − | − | − | − | − | + | − | − | − | − |
| 0 | GLY | ERY | DARA | LARA | RIB | DXYL | LXYL | ADO | MDX |
| + | + | + | + | − | − | − | − | + | + |
| GAL | GLU | FRU | MNE | SBE | RHA | DUL | INO | MAN | SOR |
| − | + | + | + | + | + | + | + | ? | − |
| MDM | MDG | NAG | AMY | ARB | ESC | SAL | CEL | MAL | LAC |
| − | + | + | − | + | − | − | − | − | − |
| MEL | SAC | TRE | INU | MLZ | RAF | AMD | GLYG | XLT | GEN |
| + | − | + | − | − | − | − | − | − | − |
| TUR | LYX | TAG | DFUC | LFUC | DARL | LARL | GNT | 2KG | 5KG |

| | % ID | T | Test against |
|---|---|---|---|
| Significant taxa | | | |
| Lactobacillus paracasei subsp. paracasei 2 | 98.5 | 0.78 | Gluconic acid (GNT) |
| | | | 83% |
| Next taxa | | | |
| Lactobacillus paracasei subsp. paracasei 1 | 1.4 | 0.55 | Lactose (LAC) 99% |
| | | | Gentiobiose (GEN) 80% |
| | | | Gluconic acid (GNT) |
| | | | 93% |

### EXAMPLE 5

### Phylogenetic Analysis of Lactobacillus paracasei subsp. paracasei SG96

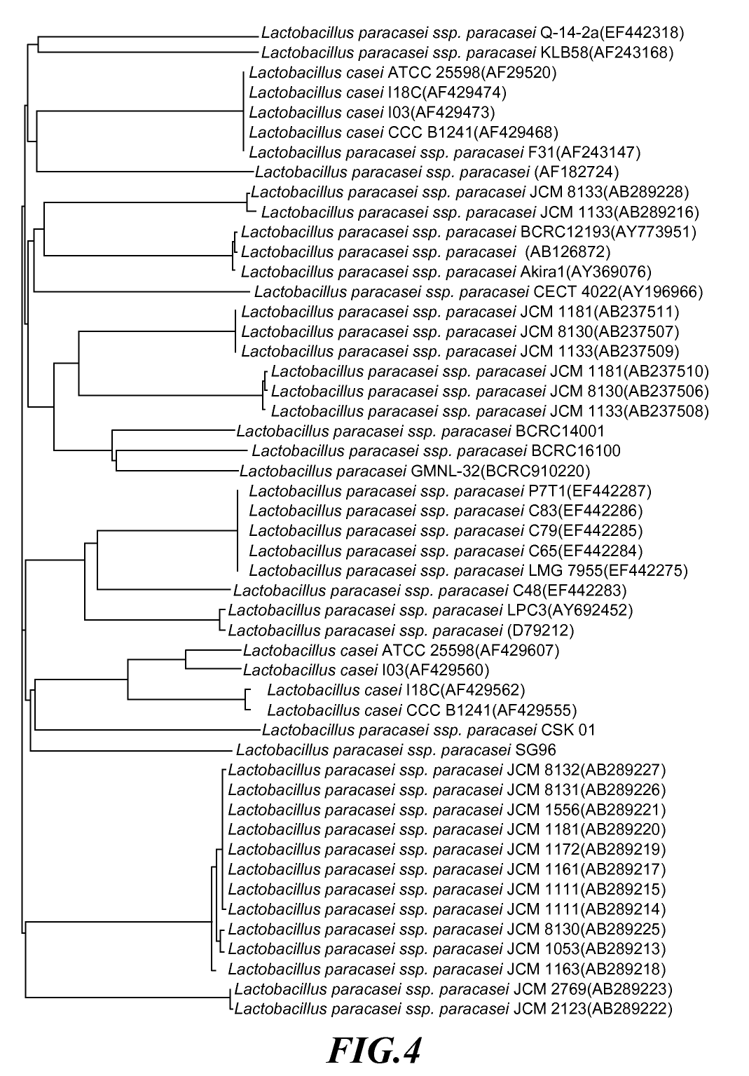

Sequences from 45 Lactobacillus paracasei strains, Lactobacillus paracasei subsp. paracasei and Lactobacillus casei published in NCBI Nucleotide Databases (http://www.ncbi.nlm.nih.gov/), sequences from 2 stains of BCRC14001 and BCRC16100 published in BCRC Databases, as well as sequences from each of strains described in ROC Patent and US Patent (GMNL-32 (BCRC910220) and CSK01), were subjected to phylogenetic analysis comparison using EMBL-EBI ClustalW2 (http://www.ebi.ac.uk/clustalw), and the results of the phylogenetic analysis were shown in FIG. 4. The result indicated that the inventive SG96 belonged to the same clone with only five bacteria strains, and one independent strain could be discriminated from these five strains. This represented that the inventive SG96 had its uniqueness in the classification system, and hence it was accurately proven that the inventive SG96 is a novel Lactobacillus paracasei subsp. paracasei microbial strain.

### EXAMPLE 6

### Inhibition Effect of Lactobacillus paracasei subsp. paracasei SG96 Strain Against Escherichia coli and Salmonella typhimurium

### 1. Disc-Agar Diffusion Assay

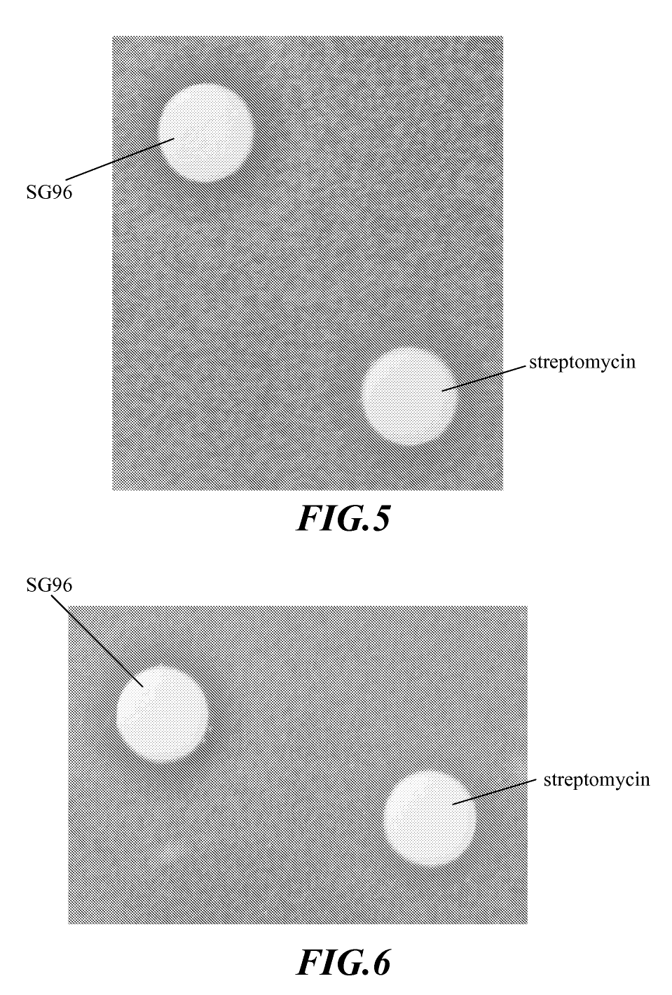

A platinum loop amounts of Escherichia coli (accession number: BCRC 11634) and Salmonella typhimurium (accession number: BCRC 12947) were inoculated respectively on slants of TSA medium (Soybean-Casein Digest Agar Medium, Difco™, REF236950), and cultured at 35-37° C. for 17-24 hours to the stationary growth phase of the strain. After determining the turbidity by a spectrophotometer, the bacteria suspensions were adjusted to a suitable turbidity (a turbidity of 80% at about 600 nm, 80% T). 0.5 ml each of the two pathogenic bacteria liquor was inoculated into a solid plate medium at about 48° C., and the mixture was poured into a Petri dish. After letting stand still for 1 hour to coagulate, a sterile filter paper tablet of 8 mm in diameter was used to pick up liquor of Lactobacillus paracasei subsp. paracasei SG96 strain, and the filter paper tablet was placed gently on the above-described Petri dish. After incubated at 35-37° C. for 17-24 hours, the result was observed and diameters of the inhibitive circle were measured. 15 g/mL of antibiotic streptomycin was used as the positive control. Results were shown in FIGS. 5 and 6.

As shown in FIGS. 5 and 6, the diameter of inhibitive circle measured from the inventive Lactobacillus paracasei subsp. paracasei SG96 strain against Escherichia coli (BCRC 11634) was 11.8 mm (see FIG. 5), while the diameter of inhibitive circle from antibiotic streptomycin was 11.0 mm. These indicated that the inventive Lactobacillus paracasei subsp. paracasei SG96 strain had a stronger inhibitory effect. Further, the diameter of inhibitive circle measured from the inventive strain against Salmonella typhimurium (BCRC 12947) was 12.5 mm (see FIG. 6), while the diameter of inhibitive circle of antibiotic streptomycin was 11.2 mm. These indicated also that the inventive Lactobacillus paracasei subsp. paracasei SG96 strain had a stronger inhibitory effect.

2. Mixed Culture Experiment of Lactobacillus paracasei subsp. paracasei SG96 Strain and Pathogenic Bacteria

In this experiment, Escherichia coli (BCRC 11634) and Salmonella typhimurium (BCRC 12947) were used as pathogenic bacteria. These pathogenic bacteria were mixed and cultured with Lactobacillus paracasei subsp. paracasei SG96 strain (TSA medium, at 37° C.), and samples were taken during culturing. Samples were diluted serially to a suitable fold and aliquots each of 100 μL were coated separately on MRS and TSA Petri dishes. The MRS Petri dish was incubated at 35-37° C. under anaerobic condition for 24-48 hours, while TSA Petri dish was incubated at 35-37° C. under aerobic condition for 24 hours. After colonies were formed, bacteria counts of Lactobacillus paracasei subsp. paracasei SG96 strain (MRS Petri dish) and pathogenic bacteria (TSA Petri dish) were determined.

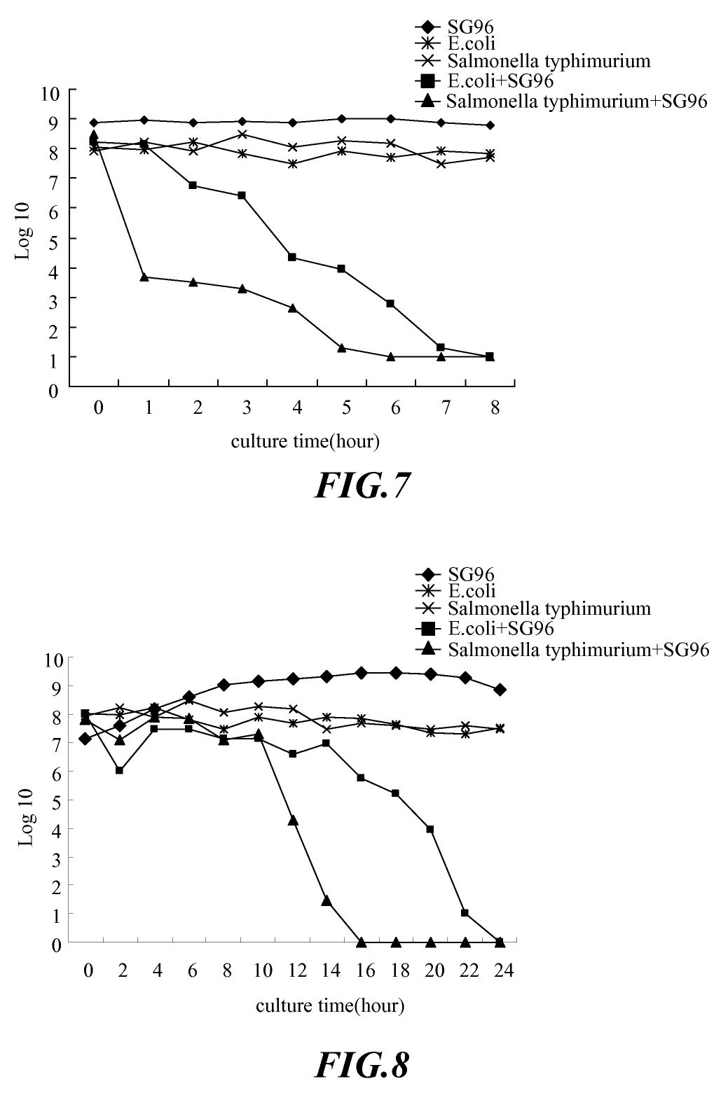

As shown in FIG. 7, when Lactobacillus paracasei subsp. paracasei SG96 strain (10⁸ CFU/ml) was mixed and cultured with Escherichia coli (BCRC 11634) and Salmonella typhimurium (BCRC 12947), respectively, the bacteria count of Escherichia coli was decreased form 10⁸ to 10¹ within 8 hours, while the bacteria count of Salmonella typhimurium was decreased from 10⁸ to 10¹ within 6 hours. These indicated that the inventive strain had a strong inhibitory effect, and even had an effect of killing bacteria.

As shown in FIG. 8, when Lactobacillus paracasei subsp. paracasei SG96 strain (10⁷ CFU/ml) was mixed and cultured with Escherichia coli (BCRC 11634) and Salmonella typhimurium (BCRC 12947), respectively, the bacteria count of Escherichia coli was decreased form 10⁸ to 0 within 22 hours, while the bacteria count of Salmonella typhimurium was decreased from 10⁸ to 0 within 16 hours.

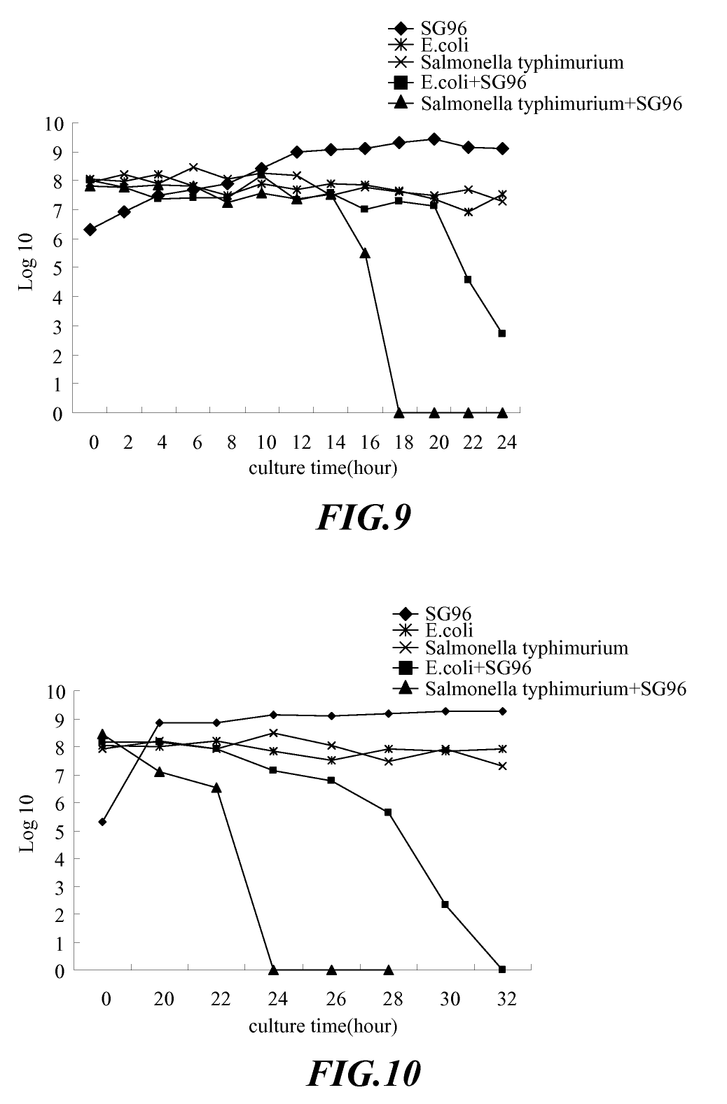

As shown in FIG. 9, when Lactobacillus paracasei subsp. paracasei SG96 strain (10⁶ CFU/ml) was mixed and cultured with Escherichia coli (BCRC 11634) and Salmonella typhimurium (BCRC 12947), respectively, the bacteria count of Escherichia coli was decreased form 10⁸ to 10² within 24 hours, while the bacteria count of Salmonella typhimurium was decreased from 10⁸ to 0 within 18 hours.

As shown in FIG. 10, when Lactobacillus paracasei subsp. paracasei SG96 strain (10⁵ CFU/ml) was mixed and cultured with Escherichia coli (BCRC 11634) and Salmonella typhimurium (BCRC 12947), respectively, the bacteria count of Escherichia coli was decreased form 10⁸ to 0 after 22 hours, while the bacteria count of Salmonella typhimurium was decreased from 10⁸ to 0 within 24 hours.

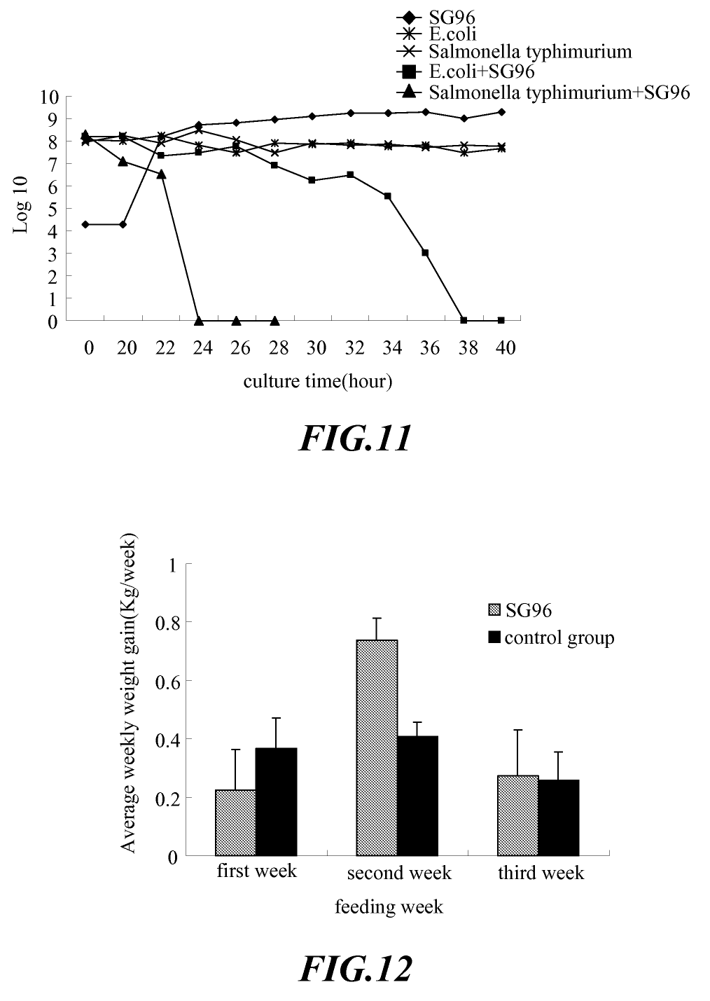

As shown in FIG. 11, when Lactobacillus paracasei subsp. paracasei SG96 strain (10⁴ CFU/ml) was mixed and cultured with Escherichia coli (BCRC 11634) and Salmonella typhimurium (BCRC 12947), respectively, the bacteria count of Escherichia coli was decreased form 10⁸ to 0 after 38 hours, while the bacteria count of Salmonella typhimurium was decreased from 10⁸ to 0 within 24 hours.

### EXAMPLE 7

### Animal Test

1. The Effect of Lactobacillus paracasei subsp. paracasei SG96 Strain on the Body Weight of Natal Un-Weaned Piglet

A dairy farmer selected natal un-weaned piglets born from the same sow at the same time, and divided them into two groups. One group was treated as a test group and was administered only an aqueous solution containing 1*10⁷ CFU/mL of Lactobacillus paracasei subsp. paracasei SG96 strain, and the other group was treated as a control group by feeding antibiotic, oxytetracycline (OTC) alone, for a total feeding time of 21 days. During this period, weight changes of the piglet were recorded every week, and statistical analysis was performed by T-test. Results shown in FIG. 12. After feeding with Lactobacillus paracasei subsp. paracasei SG96 strain for two weeks, the resulting weight gain rate was more remarkable than that of the control group (p<0.05), wherein the average weekly weight gain of the test group was 0.7-0.8 Kg. After feeding with Lactobacillus paracasei subsp. paracasei SG96 strain for three weeks, the total weight gain of piglets in the test group was also higher than that of the control group. These indicated that feeding with Lactobacillus paracasei subsp. paracasei SG96 strain could promote the weight gain of the un-weaned piglet, and improved feed conversion rate of feed for the piglet.

2. The Effect of Reducing Diarrhea for Natal Un-Weaned Piglet Using Lactobacillus paracasei subsp. paracasei SG96 Strain

Since natal un-weaned piglets are susceptible to diarrhea that is commonly caused by sow nursing or environmental factors, piglets often die early due to serious diarrhea. It is therefore desirable for normal growth of piglets if the diarrhea condition can be improved earlier.

A dairy farmer selected natal un-weaned piglets born from the same sow at the same time, and divided them into two groups. One group was treated as a test group and was administrated only with aqueous solution containing 1*10⁷ CFU/mL of Lactobacillus paracasei subsp. paracasei SG96 strain, and the other group was treated as a control group by feeding antibiotic, oxytetracycline (OTC) alone, for a total feeding time of 21 days. During the period, diarrhea conditions of every piglet was recorded every week and gave a corresponding score according to the standard described in the literature published by Underdahl et al. (Underdahl N R, Torres-Medina A, Dosten A R. 1982. Effect of Streptococcus faecium C-68 in control of Escherichia coli-induced diarrhea in gnotobiotic pigs. Am. J. Vet. Res. 1982.12: 2227-2232). The score was given as follows:

0=normal;

1=soft stool, dry around anus;

2=moist around anus;

3=watery diarrhea;

4=standard 1-3+bad appetite, weight lost and emaciated.

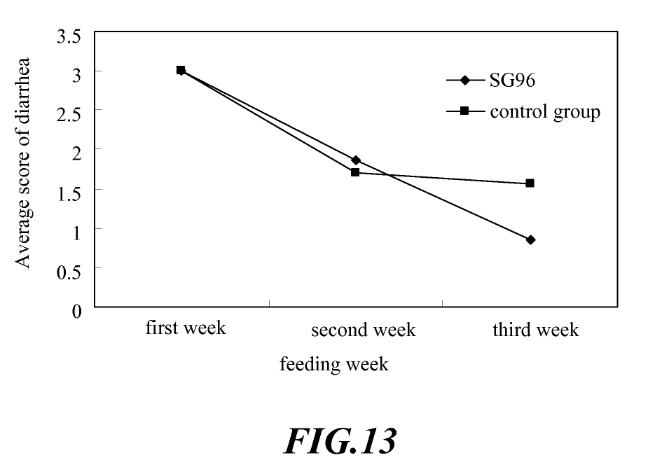

A statistical analysis was performed by T-test, and the result was shown in FIG. 13. Test group fed with Lactobacillus paracasei subsp. paracasei SG96 strain demonstrated an effect of reducing diarrhea, with the effect similar to that of the control group at the second week. After feeding for three weeks, the effect of reducing diarrhea by feeding with Lactobacillus paracasei subsp. paracasei SG96 strain was better than that of the control group (the group fed with antibiotics), and differed significantly therebetween (p<0.05). These indicated that the inventive Lactobacillus paracasei subsp. paracasei SG96 strain exhibited good anti-diarrhea effect, and the performance was better than that obtained from antibiotics commonly used by dairy farmers, and could achieve the purpose of leaving no residual antibiotics in the body of the piglet.

In view of the foregoing, the novel Lactobacillus paracasei subsp. paracasei SG96, a bacteriostatic composition containing the same and use thereof provided by the invention has the following advantages:

1. The inventive Lactobacillus paracasei subsp. paracasei SG96 strain has been confirmed through Gram stain analysis, API 50 CHL analysis, 16S rDNA sequencing analysis, phylogenetic analysis, to be different in phylogenetic relation from conventional Lactobacillus paracasei BCRC 910220, and Lactobacillus paracasei subsp. paracasei BCRC14001, and BCRC16100, and is a novel Lactobacillus paracasei subsp. paracasei.

2. When the inventive Lactobacillus paracasei subsp. paracasei SG96 strain is subjected to disc-agar diffusion analysis and mixed culture experiment analysis with pathogenic bacteria, respectively, results indicate that the inventive SG96 strain not only can inhibit the growth of pathogenic bacteria such as Escherichia coli, Salmonella typhimurium and the like, but also its bacteriostatic effect is stronger that that of antibiotic streptomycin, and even has an effect of killing bacteria.

3. When the inventive Lactobacillus paracasei subsp. paracasei SG96 strain is formulated into aqueous solution and fed to un-weaned piglets, results show that the inventive SG96 strain can promote remarkably the weight gain of un-weaned piglet, improve the feed conversion rate of the feed for the piglet, and has a good anti-diarrhea effect with a performance better than that obtained using conventional antibiotics. These prove that the inventive SG96 strain possesses an ability of enhancing the pathogenic bacteria-resistance of animals.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A novel Lactobacillus paracasei subsp. paracasei SG96 strain, which has been deposited in China General Microbiological Culture Collection Center (CGMCC), with accession number: CGMCC 2697.

2. A composition comprising Lactobacillus paracasei subsp. paracasei SG96 strain of claim 1.

3. A composition as recited in claim 2, wherein said composition is used for inhibiting the growth of Escherichia coli.

4. A composition as recited in claim 2, wherein said composition is used for inhibiting the growth of Salmonella typhimurium.

5. A composition as recited in claim 2, wherein said composition is used for inhibiting diarrhea.

6. A composition as recited in claim 2, wherein said composition is used as an additive in animal drinking water, as an additive in animal feed, as an medical composition for animal and human, as an additive in food product, as an additive in beverage, and as a food product, beverage, and health food products.
